(19) United States
(12) Patent Application Publication
(10) Pub. No.: US 2025/0204919 A1
(43) Pub. Date: Jun. 26, 2025

(54) IMPLANTABLE MEDICAL DEVICES WITH SHAPE MEMORY POLYMER ANCHORS

(71) Applicant: BOSTON SCIENTIFIC SCIMED, INC., MAPLE GROVE, MN (US)

(72) Inventor: Patrick Willoughby, Shoreview, MN (US)

(73) Assignee: BOSTON SCIENTIFIC SCIMED, INC., MAPLE GROVE, MN (US)

(21) Appl. No.: 18/987,195

(22) Filed: Dec. 19, 2024

Related U.S. Application Data
(60) Provisional application No. 63/612,493, filed on Dec. 20, 2023.
(60) Provisional application No. 63/612,507, filed on Dec. 20, 2023.
(60) Provisional application No. 63/612,569, filed on Dec. 20, 2023.
(60) Provisional application No. 63/612,582, filed on Dec. 20, 2023.
(60) Provisional application No. 63/561,406, filed on Mar. 5, 2024.
(60) Provisional application No. 63/561,415, filed on Mar. 5, 2024.
(60) Provisional application No. 63/560,160, filed on Mar. 1, 2024.
(60) Provisional application No. 63/560,174, filed on Mar. 1, 2024.

(51) Int. Cl. A61B 17/12; A61B 17/00

(52) U.S. Cl. CPC A61B 17/12122; A61B 17/12031; A61B 2017/00862; A61B 2017/00867; A61B 2017/00942; A61B 2017/00946

(57) ABSTRACT

An implantable medical device includes a device body and an anchor secured relative to the device body. The anchor is adapted to secure the device body in a desired location. The anchor includes a shape memory polymeric component, where the shape memory polymeric component has a first configuration for initial implantation and has a second configuration subsequent to initial implantation. The shape memory polymeric component may include a shape memory foam. The shape memory polymeric component may include a shape memory polymer.

### CROSS-REFERENCE TO RELATED APPLICATIONS

This application claims the benefit of priority of U.S. Provisional Application No. 63/612,493 filed Dec. 20, 2023, 63/612,507, filed Dec. 20, 2023, 63/612,569, filed Dec. 20, 2023, 63/612,582, filed Dec. 20, 2023, 63/561,406, filed Mar. 5, 2024, 63/561,415, filed Mar. 5, 2024, 63/560,160, filed Mar. 1, 2024, and 63/560,174, filed Mar. 1, 2024, the entirety disclosure of which is hereby incorporated by reference.

### TECHNICAL FIELD

The disclosure relates generally to medical devices and more particularly to medical devices that include shape memory polymer anchors.

### BACKGROUND

A number of medical devices are deployed within various locations within the body and may include one or more anchors for securing or fixating the medical device in position. Some medical devices may be anchored to or within the heart. The anchors on these medical devices may cause issues such as acute perforation, which can lead to pericardial effusion and potential tamponade. Potential problems such as pressure necrosis, stress shielding, and chronic inflammation may lead to cardiac tissue failure. Chronic inflammation may induce thrombosis and fibrosis formation.

Medical devices implanted within the heart may include pacing leads and implantable pacemaker devices. Medical devices implanted within the heart may include left atrial appendage closure (LAAC) devices, which are intended to close off the left atrial appendage (LAA) in order to reduce the likelihood of thrombi forming in the LAA from escaping the LAA and entering the bloodstream. Thrombi that migrate through the blood vessels may eventually plug a smaller vessel downstream and thereby contribute to stroke or heart attack. Clinical studies have shown that the majority of blood clots in patients with atrial fibrillation originate in the LAA. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in an implantable medical device. The implantable medical device includes a device body and an anchor including a shape memory polymeric component, with the anchor secured relative to the device body and configured to secure the device body in a desired location. The shape memory polymeric component has a first configuration for initial implantation and has a second configuration subsequent to initial implantation.

Alternatively or additionally, the shape memory polymeric component may include a shape memory foam.

Alternatively or additionally, the shape memory polymeric component may include a shape memory polymer.

Alternatively or additionally, the shape memory polymeric component may form the anchor.

Alternatively or additionally, the shape memory polymeric component may be disposed on the anchor.

Alternatively or additionally, the anchor may further include a removable insertion aid.

Alternatively or additionally, the shape memory polymeric component may change from the first configuration to the second configuration as a result of a temperature increase of the shape memory polymeric component.

Alternatively or additionally, the shape memory polymeric component may change from the first configuration to the second configuration as a result of hydration.

Alternatively or additionally, the shape memory polymeric component may change from the first configuration to the second configuration as a result of active actuation.

Another example may be found in an implantable medical device. The implantable medical device includes a device body and an anchor including a shape memory foam component, with the anchor secured relative to the device body and configured to secure the device body in a desired location. The shape memory foam component has a compressed configuration for initial implantation and expands into an expanded configuration after implantation.

Alternatively or additionally, the shape memory foam component in its compressed configuration may form the anchor.

Alternatively or additionally, the shape memory foam component may be compressed into a shape of a helical screw in its compressed configuration.

Alternatively or additionally, the shape memory foam component may be adapted to accommodate an insertion aid therein, and the anchor may be adapted to be inserted into tissue with the insertion aid positioned within the shape memory foam component.

Alternatively or additionally, the shape memory foam component may be compressed onto the anchor.

Another example may be found in an implantable medical device. The implantable medical device includes a device body and a shape memory anchor that is secured relative to the device body and is configured to secure the device body in a desired location. The shape memory anchor has a first configuration for initial implantation and a second configuration after implantation.

Alternatively or additionally, the first configuration may include a linear configuration and the second configuration may include a curved configuration.

Alternatively or additionally, the shape memory anchor may include one or more hooks in the second configuration.

Alternatively or additionally, the shape memory anchor may have a T-shape or an L-shape in the second configuration.

Alternatively or additionally, the shape memory anchor may include a coil.

Alternatively or additionally, the coil may have a first pitch and/or diameter while in the first configuration and may have a second pitch and/or diameter while in the second configuration.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cross-sectional view of an LAA (left atrial appendage) with an illustrative LAAC (left atrial appendage closure) device positioned therein;

FIG. 2 is a schematic view of an illustrative implantable medical device including an anchor movable between a first configuration for implantation and a second, subsequent configuration;

FIG. 2A is a cross-sectional view taken along the line 2A-2A of FIG. 2;

FIG. 2B is a cross-sectional view taken along the line 2B-2B of FIG. 2;

FIG. 3 is a schematic view of an illustrative implantable medical device including an anchor movable between a first configuration for implantation and a second, subsequent configuration;

FIG. 4 is a schematic view of an illustrative implantable medical device including an anchor movable between a first configuration for implantation and a second, subsequent configuration;

FIGS. 5A and 5B are schematic views showing step by step how an illustrative anchor for an implantable medical device may be implanted with the assistance of a removable insertion aid;

FIG. 6 provides schematic views of illustrative anchors that may be deployed in combination with the removable insertion aid shown in FIGS. 5A and 5B;

FIG. 7A is a schematic view of an illustrative anchor shown in a first configuration;

FIGS. 7B and 7C are schematic views of the illustrative anchor of FIG. 7A, shown in a second configuration;

FIG. 8 is a schematic view of an illustrative anchor changing from a first configuration to a second configuration;

FIG. 9 is a schematic view of an illustrative anchor changing from a first configuration to a second configuration;

FIG. 10A is a schematic view of an illustrative anchor including a foam component, shown in a first configuration;

FIGS. 10B and 10C are schematic views of the illustrative anchor of FIG. 10A, shown in a second configuration;

FIG. 11 is a schematic view of an illustrative anchor changing from a first configuration to a second configuration;

FIG. 12 is a schematic view of an illustrative anchor changing from a first configuration to a second configuration;

FIGS. 13A and 13B are schematic views showing step by step how an illustrative anchor for an implantable medical device may be implanted; and

FIG. 14 provides schematic views of illustrative anchors.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The anchors described herein may be considered as having a first configuration for implantation and a second configuration subsequent to implantation. In some cases, the anchors may be formed of a foam such as a shape memory foam that is compressed into the first configuration, and then allowed to subsequently expand into the second configuration to better secure the anchor within the tissue to which the pertinent implantable medical device is being secured. In some instances, the anchors may be formed of a shape memory polymer and may be inserted while the anchor is in the first configuration. Subsequent to implantation, the anchor may transition into the second configuration. The second configuration may be a remembered configuration, for example. The shape memory polymer may transition from the first configuration to the second configuration as a result of exposure to body temperature blood. In some cases, the shape memory polymer may transition from the first configuration to the second configuration as a result of being actively actuated by application or electrical or thermal energy.

An implantable medical device may include a device body and an anchor that is secured relative to the device body and is configured to secure the device body in a desired location. The anchor may include a shape memory polymeric component that has a first configuration for initial implantation and a second configuration subsequent to initial implantation. The shape memory polymeric component may include a shape memory foam. The shape memory polymeric component may include a shape memory polymer. In some instances, the shape memory polymeric component may form the anchor. In some cases, the shape memory polymeric component may be disposed on the anchor. In some cases, the anchor may further include a removable insertion aid. In some cases, the shape memory polymeric component may change from the first configuration to the second configuration as a result of a temperature increase of the shape memory polymeric component. In some cases, the shape memory polymeric component may change from the first configuration to the second configuration as a result of hydration. In some instances, the shape memory polymeric component may change from the first configuration to the second configuration as a result of active actuation.

An implantable medical device may include a device body and an anchor that is secured relative to the device body and is configured to secure the device body in a desired location. The anchor may include a shape memory foam component that has a compressed configuration for initial implantation and that expands into an expanded configuration after implantation. In some cases, the shape memory foam component in its compressed configuration may form the anchor. In some instances, the shape memory foam component may be compressed into a shape of a helical screw in its compressed configuration. In some cases, the shape memory foam component may be adapted to accommodate an insertion aid therein, and the anchor is adapted to be inserted into tissue with the insertion aid positioned within the shape memory foam component. In some cases, the shape memory foam component may be compressed onto the anchor.

An implantable medical device may include a device body and a shape memory anchor that is secured relative to the device body in order to secure the device body in a desired location. The shape memory anchor has a first configuration for initial implantation and has a second configuration after implantation. In some cases, the first configuration may include a linear configuration and the second configuration may include a curved configuration. The shape memory anchor may include one or more hooks in the second configuration. The shape memory anchor may have a T-shape or an L-shape in the second configuration. In some cases, the shape memory anchor may include a coil. As an example, the coil may have a first pitch and/or diameter while in the first configuration and a second pitch and/or diameter while in the second configuration.

The anchors described herein may be applicable to a variety of different implantable medical devices. While the anchors are described with respect to anchoring an LAAC device within the LAA, these anchors may be used in anchoring a variety of different implantable medical devices at a desired location within the body. As an example, the anchors described herein may be used in anchoring a pacemaker lead within a chamber of the heart. As another example, the anchors described herein may be used in anchoring a leadless cardiac pacemaker (LCP) within a chamber of the heart. These are just examples, and are not intended to be limiting in any fashion.

The following figures illustrate selected components and/or arrangements of an implant for occluding the left atrial appendage, a system for occluding the left atrial appendage, and/or methods of using the implant and/or the system. It should be noted that in any given figure, some features may not be shown, or may be shown schematically, for simplicity. Additional details regarding some of the components of the implant and/or the system may be illustrated in other figures in greater detail. While discussed in the context of occluding the left atrial appendage, the implant and/or the system may also be used for other interventions and/or percutaneous medical procedures within a patient. Similarly, the devices and methods described herein with respect to percutaneous deployment may be used in other types of surgical procedures, as appropriate. In some examples, the devices may be used in a non-percutaneous procedure. Devices and methods in accordance with the disclosure may also be adapted and configured for other uses within the anatomy.

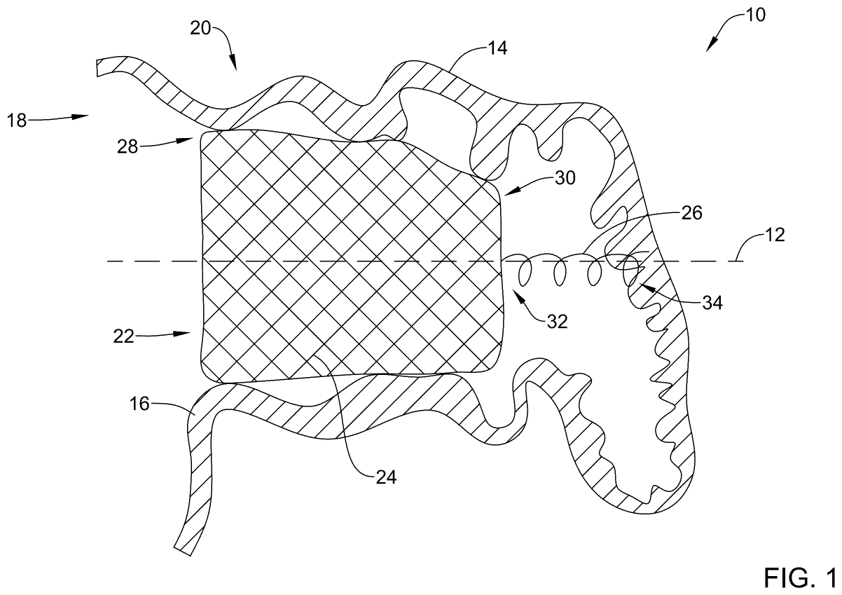

FIG. 1 is a partial cross-sectional view of a left atrial appendage 10. In some embodiments, the left atrial appendage (LAA) 10 may have a complex geometry and/or irregular surface area. It will be appreciated that the illustrated LAA 10 is merely one of many possible shapes and sizes for the LAA 10, which may vary from patient to patient. Those of skill in the art will also recognize that the medical devices, systems, and/or methods disclosed herein may be adapted for various sizes and shapes of the LAA 10, as necessary. The left atrial appendage 10 may include a generally longitudinal axis 12 arranged along a depth of a main body 20 of the left atrial appendage 10. The main body 20 may include a lateral wall 14 and an ostium 16 forming a proximal mouth 18. In some examples, a lateral extent of the ostium 16 and/or the lateral wall 14 may be smaller or less than a depth of the main body 20 along the longitudinal axis 12, or a depth of the main body 20 may be greater than a lateral extent of the ostium 16 and/or the lateral wall 14. In some examples, the LAA 10 may narrow quickly along the depth of the main body 20 or the left atrial appendage may maintain a generally constant lateral extent along a majority of depth of the main body 20. In some examples, the LAA 10 may include a distalmost region formed or arranged as a tail-like element associated with a distal portion of the main body 20. In some examples, the distalmost region may protrude radially or laterally away from the longitudinal axis 12.

FIG. 1 also shows a left atrial appendage closure (LAAC) device 22 that is disposed within the LAA 10. The LAAC device 22 may be considered as including a device body 24 and an anchor 26 that is secured relative to the device body 24. As shown, the anchor 26 is a helical anchor, but other types and shapes of anchors are contemplated. In some instances, the anchor 26 extends at least partially into the lateral wall 14 of the LAA 10, thereby helping to secure the LAAC device 22 in position relative to the LAA 10. In some cases, the device body 24 may be an expandable foam or even a shape memory foam, and may have a compressed configuration for delivery and an expanded configuration (as shown) once implanted. In some cases, the device body 24 may include a metallic or polymeric frame, with a fabric or other material forming an occlusive cover extending over at least a portion of the metallic or polymeric frame. The WATCHMAN® implant, which is commercially available from Boston Scientific, is an example of an LAAC device having a metallic frame and an occlusive covering extending over at least part of the metallic frame.

The device body 24 extends from a proximal region 28 to a distal region 30. Similarly, the anchor 26 extends from a proximal region 32 to a distal region 34. In some instances, the proximal region 32 of the anchor 26 is secured to the distal region 30 of the device body 24. In some instances, the proximal region 32 of the anchor 26 may extend proximally into the device body 24 when the device body 24 is a foam construct. In some instances, the anchor 26 may be adhesively secured to the distal region 30 of the device body 24. The anchor 26 may be formed of a shape memory foam that has been compressed or crimped into a lower profile for insertion. The anchor 26 may include a metallic or polymeric structure underlying a shape memory foam coating, for example. The anchor 26 may be formed of a shape memory polymer having an initial configuration (as shown) for implantation that will subsequently gain a new profile subsequent to implantation.

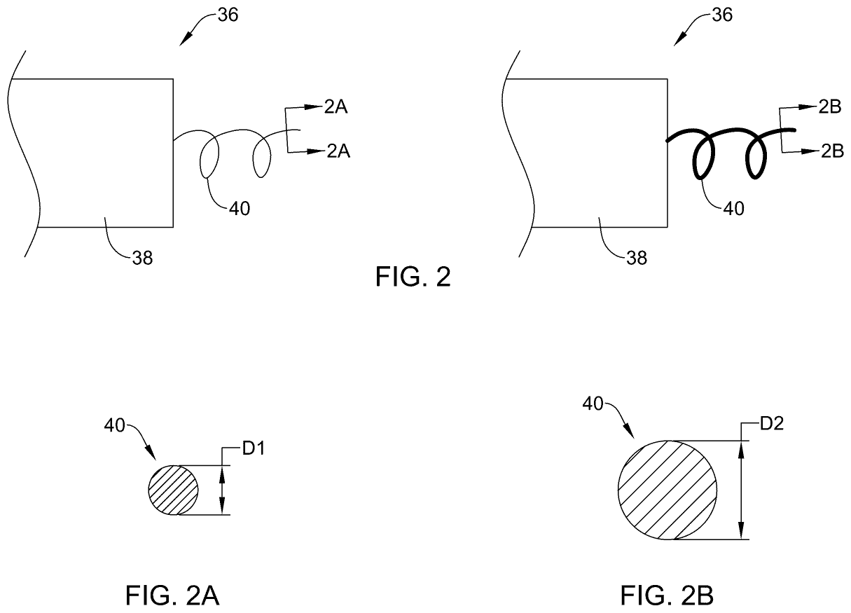

In some instances, the anchor 26 may take a variety of forms, and may itself be movable from a first configuration such as for initial implantation to a second configuration subsequent to initial implantation. FIG. 2 shows an implantable medical device 36 that includes a device body 38 (a distal portion of the device body 38 is shown) and a helical anchor 40 extending distally from the device body 38. The implantable medical device 36 may generically represent any of a variety of different implantable medical devices, such as but not limited to an LAAC device, a pacemaker lead, a leadless cardiac pacemaker, and others. In some instances, the helical anchor 40 is formed of an expandable foam or shape memory foam that has been compressed down into the profile shown on the left hand side of FIG. 2. This may be considered as being a first configuration. On the right hand side of FIG. 2, it can be seen that the helical anchor 40 has expanded into its second configuration.

As shown in FIG. 2, the material forming the helical anchor 40 expands in diameter. FIG. 2A is a cross-sectional view taken along the line 2A-2A of FIG. 2, which shows the helical anchor 40 in its first configuration. As shown, the helical anchor 40 has a cross-sectional diameter D₁ in its first configuration. FIG. 2B is a cross-sectional view taken along the line 2B-2B of FIG. 2, which shows the helical anchor 40 in its second configuration. As shown, the helical anchor 40 has a cross-sectional diameter D₂ in its second configuration. In some cases, the cross-sectional diameter D₂ may be at least fifty percent larger than the cross-sectional diameter D₁, or sixty percent larger, or seventy percent larger, or eighty percent larger, or ninety percent larger, or one hundred percent larger, or even more than one hundred percent larger, than the cross-sectional diameter D₁. While the cross-sectional views shown in FIGS. 2A and 2B show that the helical anchor 40 is formed of a single material, in some instances the helical anchor 40 may include a foam component overlaid on an underlying support structure. In some cases, having the cross-sectional diameter increase when going from the first configuration to the second configuration may help the helical anchor 40 to better grip the tissue into which the helical anchor 40 is extending. While the cross-sectional views shown in FIGS. 2A and 2B show that the helical anchor has a circular cross-section, in some cases the helical anchor may have a non-circular cross-section (e.g., trigonal, polygonal, rectilinear, etc.).

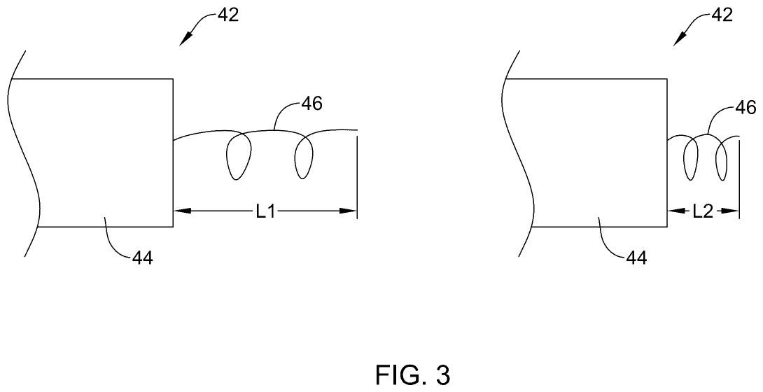

FIG. 3 shows an implantable medical device 42 that includes a device body 44 (a distal portion of the device body 44 is shown) and a helical anchor 46 extending distally from the device body 44. The implantable medical device 42 may generically represent any of a variety of different implantable medical devices, such as but not limited to an LAAC device, a pacemaker lead, a leadless cardiac pacemaker, and others. In some instances, the helical anchor 46 is formed of an expandable foam or shape memory foam that has been compressed down into the profile shown on the left hand side of FIG. 3. This may be considered as being a first configuration. On the right hand side of FIG. 3, it can be seen that the helical anchor 46 has expanded into its second configuration.

As shown in FIG. 3, the material forming the helical anchor 46 is adapted to reduce in pitch when in its second configuration. As shown on the left hand side of FIG. 3, the helical anchor 46 has an overall length (exterior to the device body 44) L₁ in its first configuration and the helical anchor 46 has an overall length L₂ in its second configuration. The overall length L₁ may be at least fifty percent larger than the overall length L₂, or sixty percent larger, or seventy percent larger, or eighty percent larger, or ninety percent larger, or one hundred percent larger, or even more than one hundred percent larger, than the overall length L₁. In some cases, having the overall length (or pitch) decrease when going from the first configuration to the second configuration may help the helical anchor 46 to better grip the tissue into which the helical anchor 46 is extending. While FIG. 2 shows the helical anchor 40 changing in its cross-sectional diameter, and FIG. 3 shows the helical anchor 46 changing in its overall length (or pitch), it will be appreciated that a helical anchor may change in both cross-sectional diameter and in overall length when changing from the first configuration to the second configuration.

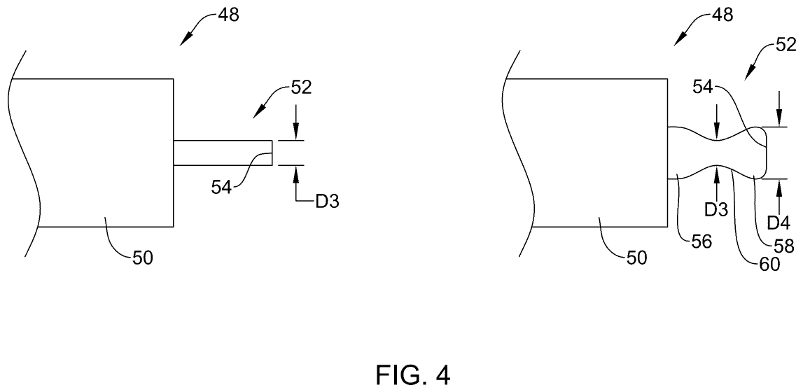

FIG. 4 shows an implantable medical device 48 that includes a device body 50 (a distal portion of the device body 50 is shown) and an anchor 52 extending distally from the device body 50. The implantable medical device 48 may generically represent any of a variety of different implantable medical devices, such as but not limited to an LAAC device, a pacemaker lead, a leadless cardiac pacemaker, and others. In some instances, the anchor 52 is formed of an expandable foam or shape memory foam that has been compressed down into the profile shown on the left hand side of FIG. 4. This may be considered as being a first configuration. On the right hand side of FIG. 4, it can be seen that the anchor 52 has expanded into its second configuration. In some cases, as shown, the first configuration may be a cylindrical profile that is easy to insert into tissue. In some cases, the cylindrical profile may have a blunt distal end 54. In some instances, the cylindrical profile may have a rounded-over distal end 54, or even a sharped or tapered distal end 54 to facilitate insertion of the anchor 52 into tissue. The second configuration may have a non-cylindrical shape that facilitates securing the anchor 52 with respect to the tissue into which the anchor 52 has been inserted. In some instances, as shown, in the second configuration the anchor 52 has an hourglass profile, with a radially enlarged proximal region 56, a radially enlarged distal region 58 and an intervening region 60 that is not radially enlarged relative to the first configuration.

As an example, the radially enlarged proximal region 56 may be adapted to remain on a proximal side of a tissue wall and the radially enlarged distal region 58 may be adapted to be positioned on a distal side of a tissue wall. In some cases, the intervening region 60 may accommodate the tissue wall that is positioned between the radially enlarged proximal region 56 and the radially enlarged distal region 58. The anchor 52 may have an overall diameter D₃ while in the first configuration, as shown on the left hand side of FIG. 4. The radially enlarged proximal region 56 and the radially enlarged distal region 58 may each have an overall diameter D₄ that is greater than the overall diameter D₃. In some cases, the intervening region 60 may also have an overall diameter of D₃. In some instances, the overall diameter D₄ may be at least fifty percent larger than the overall diameter D₃, or sixty percent larger, or seventy percent larger, or eighty percent larger, or ninety percent larger, or one hundred percent larger, or even more than one hundred percent larger, than the overall diameter D₃. While the radially enlarged proximal region 56 and the radially enlarged distal region 58 are shown as both having the same overall diameter (D₄), in some cases the radially enlarged proximal region 56 and the radially enlarged distal region 58 may have different overall diameters. In some cases, the radially enlarged distal region 58 may have an overall diameter that is greater than the radially enlarged proximal region 56.

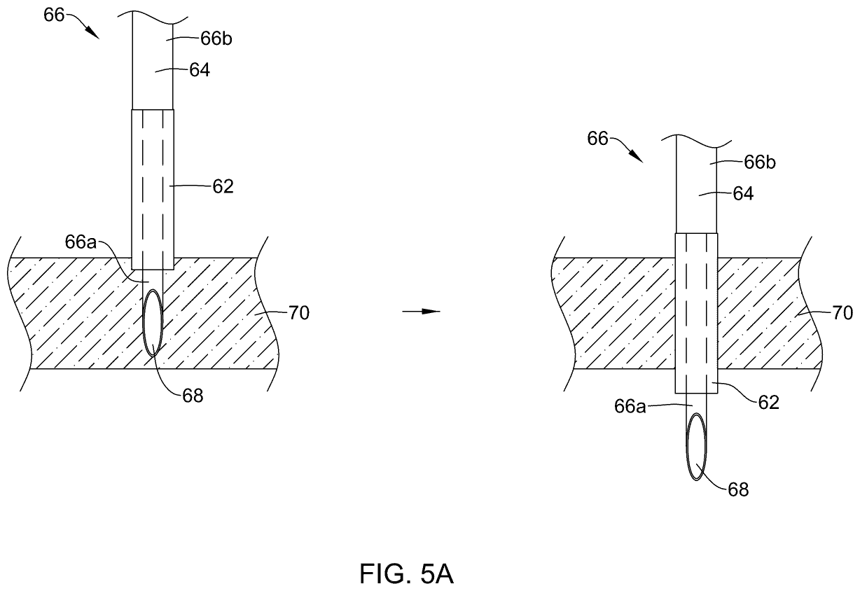

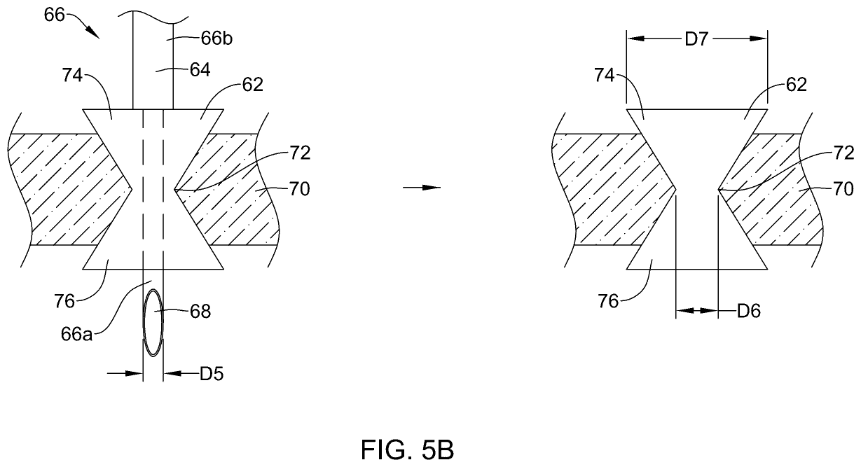

In some instances, an implantable medical device may include a foam anchor that may be inserted into tissue using a removable insertion tool. FIGS. 5A and 5B show insertion of a foam anchor 62 using a removable insertion tool 64. FIGS. 5A and 5B only show the foam anchor 62, and do not show the foam anchor 62 being attached to any device body. It will be appreciated that the foam anchor 62 will be attached to an appropriate device body, such as but not limited to the device bodies 24, 38, 44 or 50 shown in previous Figures. As shown on the left hand side of FIG. 5A, the foam anchor 62 is in a compressed configuration in which the foam anchor is secured about the removable insertion tool 64. In some cases, the removable insertion tool 64 may take the form of a straight needle having a shaft 66 having a distal tapered segment 68 that is adapted to facilitate advancement of the removable insertion tool 64 and the foam anchor 62 into tissue 70. In some cases, the shaft 66 includes a distal portion 66a having a first diameter and a proximal portion 66b having a second diameter that is greater than the first diameter. In some cases, a step-wise difference between the distal portion 66a and the proximal portion 66b is sufficient to hold the foam anchor 62 in position relative to the removable insertion tool 64.

The removable insertion tool 64 is advanced into the tissue 70. As seen on the right hand side of FIG. 5A, the removable insertion tool 64 has been advanced through the tissue 70 to a point at which the foam anchor 62 is positioned within the tissue 70. The foam anchor 62 is still in its compressed configuration for insertion.

Moving to FIG. 5B, and with respect to the left hand side of FIG. 5B, the foam anchor 62 and the removable insertion tool 64 have not moved relative to the tissue 70, and are still in the same position they were each in as shown on the right hand side of FIG. 5A. However, the foam anchor 62 has changed from its compressed configuration to an expanded configuration in which the foam anchor 62 is adapted to better anchor within the tissue 70. Finally, the removable insertion aid 64 may be withdrawn, as shown on the right hand side of FIG. 5B. In some cases, the removable insertion aid 64 may be withdrawn either before or after the foam anchor 62 has changed from its compressed configuration to its expanded configuration. The distal portion 66a of the shaft 66 has a diameter D₅. In some instances, the distal portion 66a will form an aperture extending through the tissue 70 having the same diameter. In its expanded configuration, the foam anchor 62 has a minimum diameter D₆ at or near a center point 72 of the foam anchor 62, and has a maximum diameter D₇ for each of a proximal region 74 and a distal region 76.

In some cases, the minimum diameter D₆ of the foam anchor 62 may be at least five percent larger than the diameter D₅. In some cases, minimum diameter D₆ of the foam anchor 62 may be at least ten percent larger or at least twenty percent larger or at least thirty percent larger, or more, than the diameter D₅. In some instances, the maximum diameter D₇ may be at least fifty percent larger than the minimum diameter D₆, or sixty percent larger, or seventy percent larger, or eighty percent larger, or ninety percent larger, or one hundred percent larger, or even more than one hundred percent larger, than the minimum diameter D₆.

While a step-wise change in diameter of the shaft 66 is one way to hold the foam anchor 62 in position relative to the removable insertion tool 64, it is not the only way. For example, in some cases the foam anchor 62 may be crimped directly onto the shaft 66, which would provide a compressive force between the foam anchor 62 and the shaft 66. Once the foam anchor 62 expands, the stiffness of the foam would drop significantly, removing the compressive force that was holding the foam anchor 62 in position relative to the shaft 66. As an example, when compressed, the foam anchor 62 may exert a force of up to about 50 Newtons on the shaft 66. Once expanded, the foam anchor 62 may only exert a force of about 5 Newtons on the shaft 66, which will allow the shaft 66 to be withdrawn.

In some instances, a second piece of shape memory foam or shape memory polymer may hold the foam anchor 62 in position relative to the shaft 66 until such time as the foam anchor 62 and the second piece of shape memory foam or shape memory polymer expands, thereby releasing the foam anchor 62. In some cases, the shaft 66 may include a keyway or other mechanism that engages the foam anchor 62. In some cases, the second piece of shape memory foam or shape memory polymer may form the keyway or other mechanism. Withdrawing the keyway or other mechanism after the foam anchor 62 has been positioned within tissue may allow withdrawal of the shaft 66. In some cases, an active mechanism (not shown) may be used to temporarily hold the foam anchor 62 in position on the shaft 66.

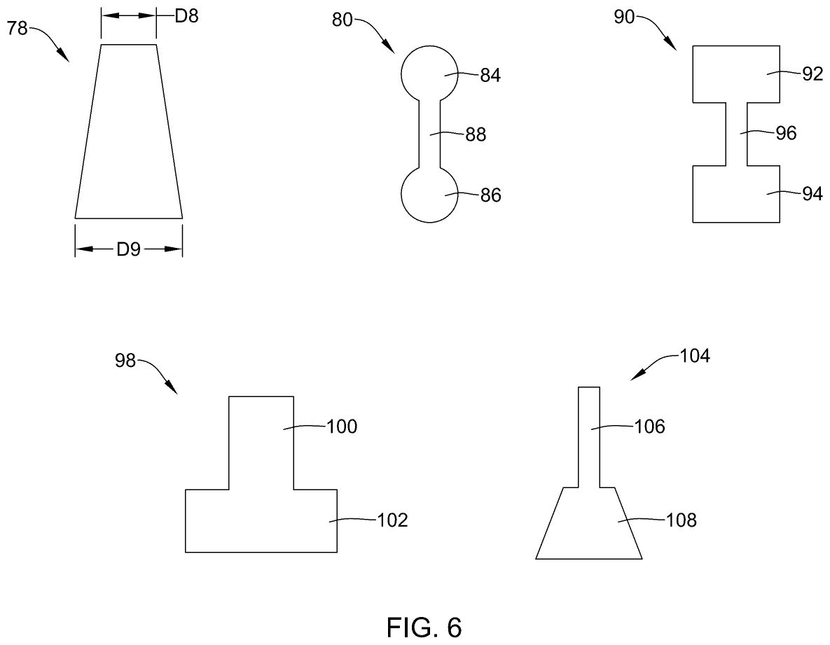

The profile shown in FIGS. 5A and 5B for the expanded configuration of the foam anchor 62 is just one example of a possible profile. FIG. 6 provides a number of additional examples of possible profiles for the foam anchor 62. FIG. 6 includes a frustoconical profile 78 that monotonically increases from a minimum diameter D₈ to a maximum diameter D₉. In some instances, the maximum diameter D₉ may be about twice the minimum diameter D₈. In some instances, the minimum diameter D₈ may range from about ten percent of the maximum diameter D₉ to about eighty percent of the maximum diameter D₉. FIG. 6 includes a barbell profile 80 that includes a first ball-shaped region 84, a second ball-shaped region 86 and an intervening cylindrical region 88, where the first ball-shaped region 84 and the second ball-shaped region 86 each have a diameter greater than that of the intervening cylindrical region 88. As an example, the first ball-shaped region 84 and the second ball-shaped region 86 may each have a diameter that is about twice that of the intervening cylindrical region 88.

FIG. 6 includes a profile 90 having a first disk-shaped region 92, a second disk-shaped region 94 and an intervening cylindrical region 96. While the first disk-shaped region 92 and the second disk-shaped region 94 may appear to be rectilinear, it will be appreciated that this is a two dimensional rendition of a three dimensional shape. In some cases, the first disk-shaped region 92 and the second disk-shaped region 94 may each have a diameter that is two to six times a diameter of the intervening cylindrical region 96. FIG. 6 includes a profile 98 having a cylindrical region 100 and a disk-shaped region 102, given that this is a two dimensional rendition of a three dimensional shape. FIG. 6 also includes a profile 104 that includes a cylindrical region 106 and a frustoconical region 108. Any of the profiles 78, 80, 90, 98 and 104 may be used as the foam anchor 62 (FIGS. 5A and 5B). In some instances, features of two or more of the profiles 78, 80, 90, 98 and 104 may be combined in forming a foam anchor.

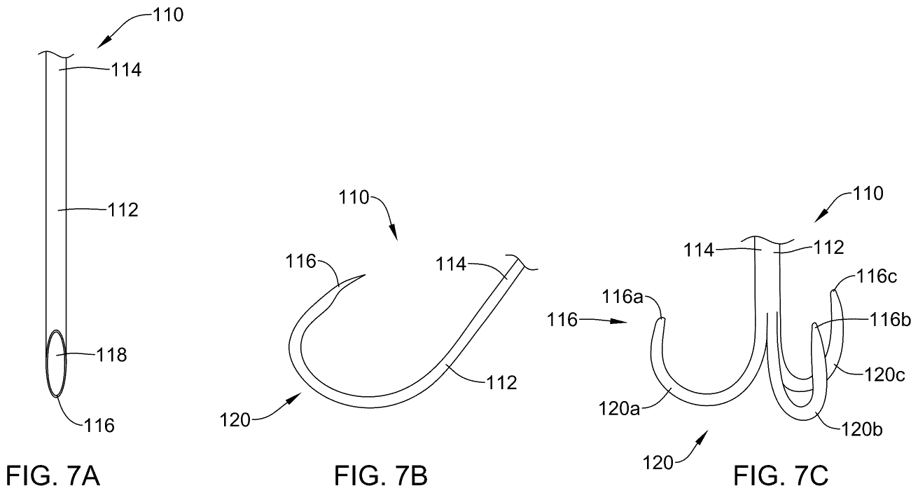

FIGS. 7A, 7B and 7C together show examples of an illustrative anchor 110 that may be made of a shape memory material. In some cases, the illustrative anchor 110 may be formed of a shape memory polymer. FIGS. 7A, 7B and 7C only show the anchor 110, and do not show the anchor 110 being attached to any device body. It will be appreciated that the anchor 110 will be attached to an appropriate device body, such as but not limited to the device bodies 24, 38, 44 or 50 shown in previous Figures.

In some cases, the anchor 110 may have an initial configuration in which the anchor 110 forms a single solid object. The anchor 110 may change or deform into a second configuration in which the anchor 110 now presents one or more additional anchor geometries. The number of additional anchor geometries may vary. In some cases, the anchor 110 may include one to four anchor geometries in the second configuration. Each of the one or more anchor geometries may be disposed equally or unequally about the anchor 110. Each of the one or more anchor geometries may expand or change from a 0 degree position (aligned with the anchor 110) to a 90 degree or greater position. Once in the second configuration, the anchor geometries may provide the anchor 110 with a cross-shaped profile, for example.

As shown, the anchor 110 includes a shaft 112 that extends from a proximal region 114 to a distal region 116. The distal region 116 may include a tapered aperture that facilitates puncturing and penetrating tissue. In some instances, the distal region 116 may include a tapered end that does not include an aperture. In FIG. 7A, the anchor 110 is shown in a linear, or first configuration. In the linear or first configuration, the anchor 110 is adapted to be inserted.

FIGS. 7B and 7C show possible shapes for the anchor 110 after the anchor 110 has changed or deformed into a second configuration. This transformation occurs after the anchor 110 has been implanted. In some instances, the second configuration may be considered as being a remembered configuration, and the first configuration represents a temporary deformation from the remembered configuration. In some cases, the transformation from first configuration to second configuration may occur as a result of exposure to moisture and/or increased temperature, such as what may occur when the anchor 110 is exposed to blood within the patient. In FIG. 7B, the anchor 110 has curved into a J-shape, thereby forming a hook 120. In some instances, the hook 120 will resist movement of the anchor 110 relative to the tissue better than the anchor 100 in its linear or first configuration. In FIG. 7C, the anchor 110 has curved into a more complex shape including several J-shaped curves each forming a hook 120, individually labeled as 120a, 120b and 120c. The hooks 120 may be considered as being anchor geometries, as referenced above. The distal region 116 of the shaft 112 has divided into a distal region 116a, a distal region 116b and a distal region 116c. In some cases, having more than one J-shaped curves, or hooks, can facilitate a more secure anchoring within the tissue.

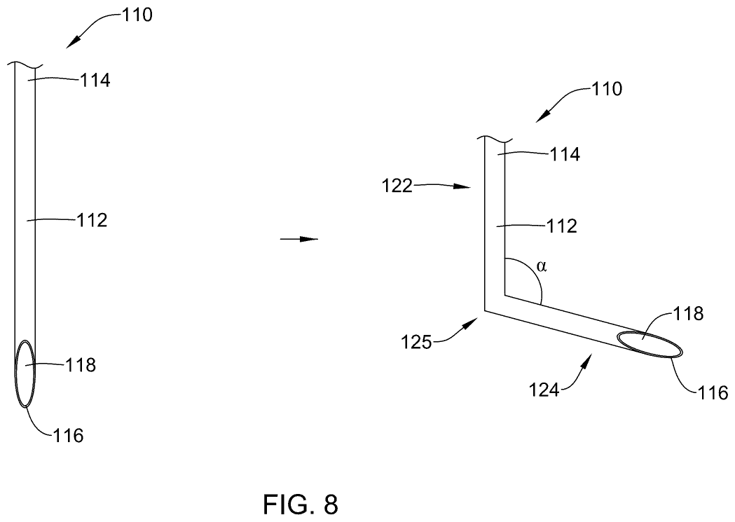

FIG. 8 shows another possible shape for the anchor 110 after the anchor 110 has changed or deformed into its second configuration. On the left hand side of FIG. 8, the anchor 110 is shown in its linear or first configuration. On the right hand side of FIG. 8, the anchor 110 has deformed into its second configuration. This transformation occurs after the anchor 110 has been implanted. In some instances, the second configuration may be considered as being a remembered configuration, and the first configuration represents a temporary deformation from the remembered configuration. In the second configuration, the shaft 112 of the anchor 110 can be seen as being bent at a considerable angle. The shaft 112 may be considered as including a proximal segment 122 and a distal segment 124, with a bend point 125 disposed between the proximal segment 122 and the distal segment 124. In the second configuration, the distal segment 124 may be bent at an angle α with respect to the proximal segment 122. In some cases, the angle α may range from about 45 degrees to about 135 degrees, or may range from about 60 degrees to 120 degrees, or may range from about 75 degrees to 105 degrees. In some instances, the angle α may be about 90 degrees when the anchor 110 is in its second configuration.

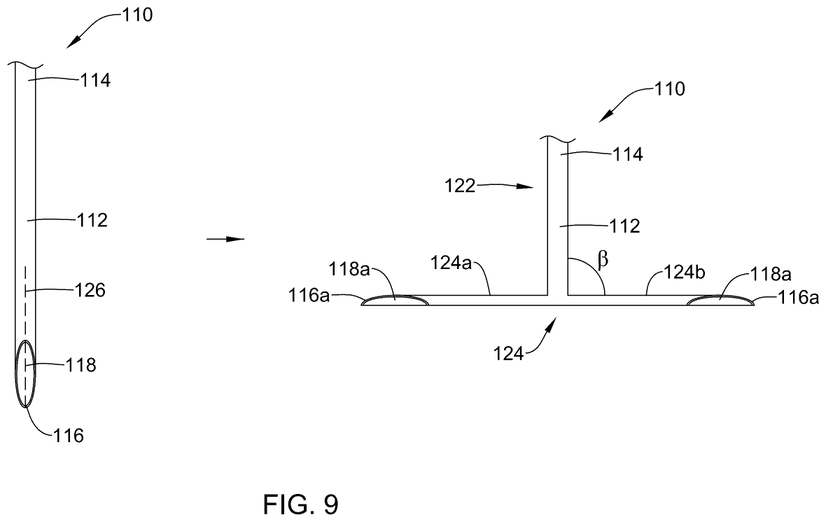

FIG. 9 shows another possible shape for the anchor 110 after the anchor 110 has changed or deformed into its second configuration. On the left hand side of FIG. 9, the anchor 110 is shown in its linear or first configuration. On the right hand side of FIG. 9, the anchor 110 has deformed into its second configuration. This transformation occurs after the anchor 110 has been implanted. In some instances, the second configuration may be considered as being a remembered configuration, and the first configuration represents a temporary deformation from the remembered configuration. In the second configuration, a distal portion of the shaft 112 of the anchor 110 can be seen as having split along a split line 126 shown in the shaft 112 on the left hand side of FIG. 9. In the second configuration, the distal segment 124 of the shaft 112 can be seen as including a distal segment portion 124a extending in a first direction with respect to the proximal shaft segment 122 and a distal segment 124b extending in a second direction with respect to the proximal shaft segment 122.

While two distal segment portions 124a and 124b are shown, it will be appreciated that the distal segment 124 may include any number of distal segment portions. In some cases, a demarcation between the proximal shaft segment 122 and the distal shaft segment 124 may be positioned such that the proximal shaft segment 122 extends through tissue and the distal segment portions 124a and 124b are positioned against a back side of the tissue, thereby increasing the holding power of the anchor 110. The distal segment portions 124a and 124b are shown as forming a right angle with respect to the proximal shaft segment 122. As shown, the distal segment portion 124b forms an angle β with respect to the proximal shaft segment 122. The distal segment portion 124a may be considered as forming a similar angle, although this is not required. In some cases, the angle β may range from about 45 degrees to about 135 degrees, or may range from about 60 degrees to 120 degrees, or may range from about 75 degrees to 105 degrees. In some cases, the angle β may be about 90 degrees, or a right angle.

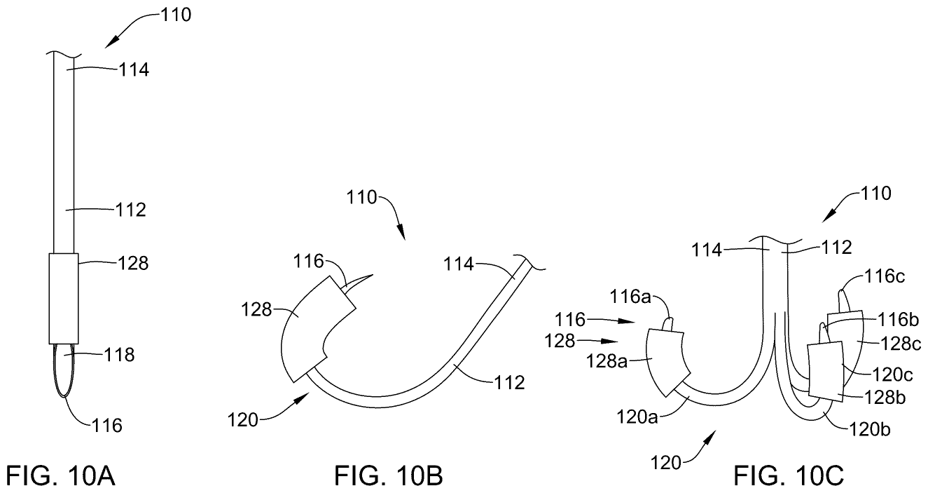

FIGS. 10A, 10B and 10C together show examples of adding a compressed foam component (or components) to the illustrative anchor 110. FIGS. 10A, 10B and 10C only show the anchor 110, and do not show the anchor 110 being attached to any device body. It will be appreciated that the anchor 110 will be attached to an appropriate device body, such as but not limited to the device bodies 24, 38, 44 or 50 shown in previous Figures. The anchor 110 includes a shaft 112 that extends from a proximal region 114 to a distal region 116. The distal region 116 may include a tapered aperture that facilitates puncturing and penetrating tissue. In some instances, the distal region 116 may include a tapered end that does not include an aperture. In FIG. 10A, the anchor 110 is shown in a linear, or first configuration. In the linear or first configuration, the anchor 110 is adapted to be inserted. The anchor 110 includes a foam component 128 that has been compressed or crimped into a compressed configuration and has been attached to the shaft 112. The foam component 128 may be secured relative to the shaft 112 by virtue of having been compressed or crimped into position. In some cases, the foam component 128 may be adhesively secured to the shaft 112. The foam component 128, as seen in FIG. 10A, may be considered as being in a compressed configuration.

FIGS. 10B and 10C show possible shapes for the anchor 110 after the anchor 110 has changed or deformed into a second configuration. This transformation occurs after the anchor 110 has been implanted. In some instances, the second configuration may be considered as being a remembered configuration, and the first configuration represents a temporary deformation from the remembered configuration. In some cases, the transformation from first configuration to second configuration may occur as a result of exposure to moisture and/or increased temperature, such as may occur when the anchor 110 is exposed to blood within the patient. In addition to the shaft 112 transforming from its first configuration to its second configuration, the foam component 128 may also expand as a result of exposure to blood, for example.

In FIG. 10B, the anchor 110 has curved into a J-shape, thereby forming a hook 120. In some instances, the hook 120 will better resist movement of the anchor 110 compared to the anchor 100 in its linear or first configuration. In FIG. 10B, it can be seen that the foam component 128 has also expanded. This can help with sealing against possible blood leaks as a result of tissue being punctured, and can also help with holding the anchor 110 in place within the tissue. In FIG. 10C, the anchor 110 has curved into a more complex shape including several J-shaped curves each forming a hook 120, individually labeled as 120a, 120b and 120c. In some cases, having more than one J-shaped curves, or hooks, can facilitate a more secure anchoring within the tissue. The foam component 128 may be seen as including a foam component 128a, a foam component 128b and a foam component 128c, with each of the foam components 128 secured relative to one of the hooks 120. Each of the foam components 128 have also expanded. This can help with sealing against possible blood leaks as a result of tissue being punctured, and can also help with holding the anchor 110 in place within the tissue. It will be appreciated that the relative location of the foam component(s) 128 is merely illustrative, as the foam component(s) 128 may be placed at any desired location relative to the shaft 112.

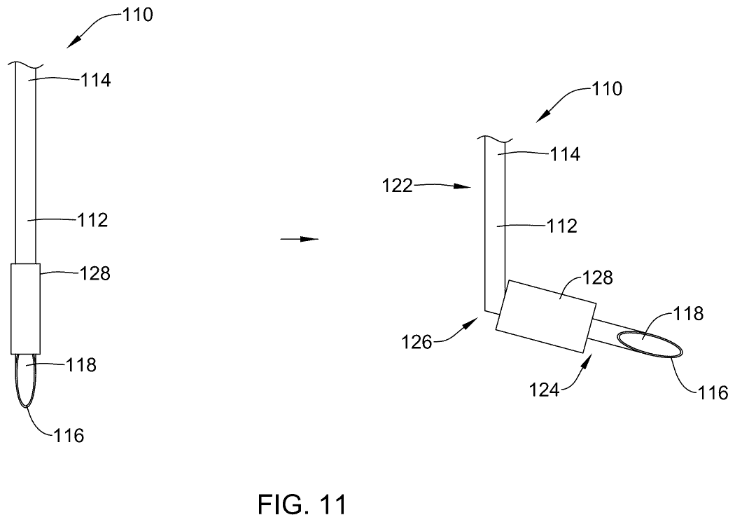

FIG. 11 shows another possible shape for the anchor 110 after the anchor 110 has changed or deformed into its second configuration. On the left hand side of FIG. 11, the anchor 110 is shown in its linear or first configuration, including the foam component 128 in its compressed configuration. On the right hand side of FIG. 11, the anchor 110 has deformed into its second configuration. This transformation occurs after the anchor 110 has been implanted. In some instances, the second configuration may be considered as being a remembered configuration, and the first configuration represents a temporary deformation from the remembered configuration. In the second configuration, the shaft 112 of the anchor 110 can be seen as being bent at a considerable angle. The shaft 112 includes the proximal segment 122 and the distal segment 124, with the bend point 126 disposed between the proximal segment 122 and the distal segment 124. The foam component 128 has also expanded. This can help with sealing against possible blood leaks as a result of tissue being punctured, and can also help with holding the anchor 110 in place within the tissue.

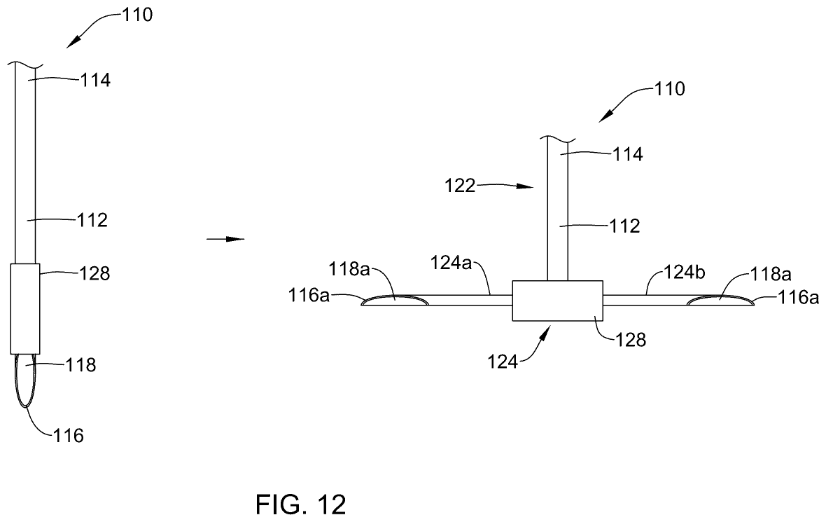

FIG. 12 shows another possible shape for the anchor 110 after the anchor 110 has changed or deformed into its second configuration. On the left hand side of FIG. 12, the anchor 110 is shown in its linear or first configuration, including the foam component 128 in its compressed configuration. On the right hand side of FIG. 12, the anchor 110 has deformed into its second configuration. This transformation occurs after the anchor 110 has been implanted. In some instances, the second configuration may be considered as being a remembered configuration, and the first configuration represents a temporary deformation from the remembered configuration. In the second configuration, a distal portion of the shaft 112 of the anchor 110 can be seen as having split along the split line 126 (visible in FIG. 9) shown in the shaft 112 on the right hand side of FIG. 9. In the second configuration, the distal segment 124 of the shaft 112 can be seen as including a distal segment portion 124a extending in a first direction with respect to the proximal shaft segment 122 and a distal segment 124b extending in a second direction with respect to the proximal shaft segment 122. The foam component 128 has also expanded. This can help with sealing against possible blood leaks as a result of tissue being punctured, and can also help with holding the anchor 110 in place within the tissue. It will be appreciated that the relative location of the foam component(s) 128 is merely illustrative, as the foam component(s) 128 may be placed at any desired location relative to the shaft 112.

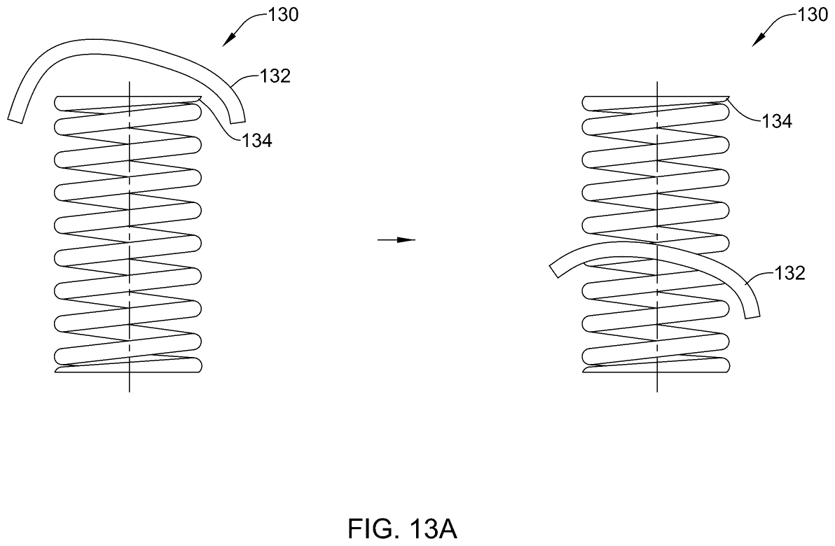

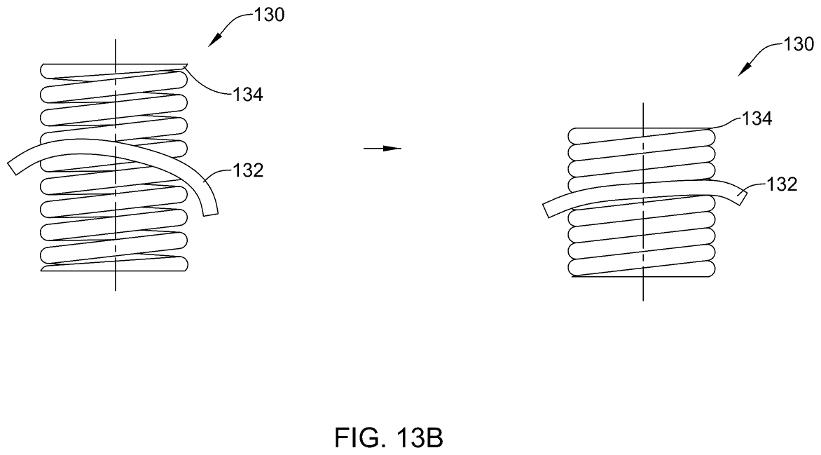

FIGS. 13A and 13B together show insertion of an anchor 130, and how the anchor 130 is advanced relative to a tissue layer 132. As an example, the tissue layer 132 may represent the lateral wall 14 of the LAA 10, but this is not required. FIGS. 13A and 13B only show the anchor 130, and do not show the anchor 130 being attached to any device body. It will be appreciated that the anchor 130 will be attached to an appropriate device body, such as but not limited to the device bodies 24, 38, 44 or 50 shown in previous Figures. In some cases, the anchor 130 may be formed of a shape memory material and thus may be changeable between a first configuration and a second configuration, where the second configuration represents a remembered configuration and the first configuration represents a temporary deformation from the remembered configuration. In some cases, the anchor 130 may be formed of a shape memory polymer. As shown, the anchor 130 is a coil having a constant diameter and a constant pitch, at least in its first configuration.

On the left hand side of FIG. 13A, the anchor 130 is in position and is about to be advanced into and through the tissue layer 132. In some instances, the anchor 130 includes a distal end 134 that is adapted to puncture the tissue layer 132 so that the anchor 130 can be advanced through the tissue layer 132 by rotating the anchor 130. On the right hand side of FIG. 13A, the anchor 130 may be seen as having been advanced through the tissue layer 132, but the anchor 130 is still in its first configuration. On the left hand side of FIG. 13B, the anchor 130 has begun transforming from its first configuration to its second configuration. As shown, the pitch of the anchor 130, or the axial distance between adjacent coil windings, is starting to be reduced. On the right hand side of FIG. 13B, the anchor 130 has completed its transformation from its first configuration to its second configuration. As the anchor 130 reduces in pitch, and collapses down onto the tissue layer 132, the anchor 130 will hold more tightly against the tissue layer 132, and thus increased force will be needed to rotate the anchor 130. This helps to secure the anchor 130 in position.

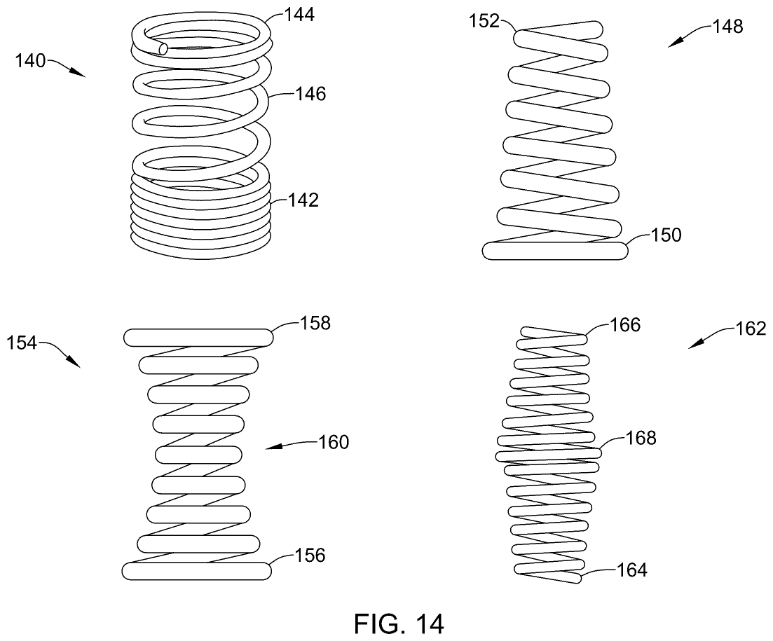

The anchor 130 shown in FIGS. 13A and 13B represents just one example of a sample coil that may be used. FIG. 14 provides a number of additional examples of possible coils that may be used as the anchor 130. In some cases, each of the coils shown in FIG. 14 represent second configurations. In some cases, each of the coils shown in FIG. 14 may have a cylindrical and/or constant pitch first configuration. By changing from a regular shape and/or constant pitch to a variable diameter and/or variable pitch, this increases the resistance of the coil from being unscrewed or otherwise loosened from tissue.

FIG. 14 includes a variable pitch coil 140 that includes a first reduced pitch region 142, a second reduced pitch region 144 and an intervening open pitch region 146. In some cases, the variable pitch coil 140 as shown represents a second configuration. The open pitch region 146 may allow for translation of the variable pitch coil 140 relative to a tissue layer. In some instances, the diameter of a coil may vary, especially in the second configuration. FIG. 14 includes a conical coil 148 in which the diameter of the coil 148 decreases smoothly from a distal region 150 to a proximal region 152. FIG. 14 includes an hourglass-shaped coil 154 having a maximum diameter at a distal region 156 and at a proximal region 158. The diameter smoothly decreases moving from either the distal region 156 or the proximal region 158 to an intermediate region 160. FIG. 14 includes a barrel-shaped coil 162 having a minimum diameter at a distal region 164 and at a proximal region 166. The diameter smoothly increases moving from either the distal region 164 or the proximal region 166 towards an intermediate region 168.

The expandable foam may include any suitable material, such as a suitable polymeric material, that is capable of transitioning from an initial configuration to an expanded configuration upon being subjected to a specific temperature or temperature range and/or exposure to moisture, and provide a suitable density in the expanded configuration for use inside of the left atrial appendage to provide an occlusive benefit without negatively impacting surrounding anatomy. In some instances, the expandable foam may be a shape memory foam. Suitable transition temperatures may be, for example, at or below about 37° C. (98.6° F.), which allows the shape memory foam to assume an initial configuration prior to and during delivery through a delivery catheter or other delivery device, and an expanded configuration for occlusion after delivery and release within the left atrial appendage, allowing the shape memory foam to be exposed to body temperature blood within the left atrial appendage. A suitable density of the shape memory foam in the expanded configuration is a density that allows the expanded configuration to be pliable and compliant and substantially conform to the left atrial appendage anatomy to create a seal to protect against the formation and escape of blood clots while having sufficient radial force to seal the left atrial appendage but not damage or impact surrounding anatomy. In some instances, the density of the shape memory foam in the expanded configuration will be from about 10 kg/m³ (about 0.62 lb/ft³) to about 1000 kg/m³ (about 62.31 lb/ft³), including from about 10 kg/m³ to about 500 kg/m³ (about 31.2 lb/ft³) including from about 10 kg/m³ to about 200 kg/m³ (about 12.5 lb/ft³), including from about 20 kg/m³ to about 100 kg/m³ (about 6.2 lb/ft³).

Generally, the material for constructing the shape memory foam is a polymeric material that is both biocompatible and substantially biostable. In some instances, biocompatibility will include meeting or surpassing the requirements of established standards for implant materials defined in ISO 10993 and USP Class VI. Substantially biostable materials include those materials that do not resorb over the intended lifetime of the medical device (such as five years, or ten years, or longer), as well as those materials that resorb slowly such that void volume is replaced by a stable tissue-like material over a period of a few months to a year.

In some instances, the shape memory foam may include a natural and/or synthetic material. Suitable natural materials may include, for example, extracellular matrix (ECM) biopolymers such as collagen, fibronectin, hyaluronic acid and elastin, non-ECM biomaterials such as cross-linked albumin, fibrin, and inorganic bioceramics such as hydroxyapatite and tricalcium phosphate. Suitable synthetic materials may include, for example, biostable polymers such as saturated and unsaturated polyolefins including polyethylene, polyacrylics, polyacrylates, polymethacrylates, polyamides, polyimides, polyurethanes, polyureas, polyvinyl aromatics such as polystyrene, polyisobutylene copolymers and isobutylene-styrene block copolymers such as styrene-isobutylene-styrene tert-block copolymers (SIBS), polyvinylpyrolidone, polyvinyl alcohols, copolymers of vinyl monomers such as ethylene vinyl acetate (EVA), polyvinyl ethers, polyesters including polyethylene terephthalate, polyacrylamides, polyethers such as polyethylene glycol, polytetrahydrofuran and polyether sulfone, polycarbonates, silicones such as siloxane polymers, and fluoropolymers such as polyvinylidene fluoride, and mixtures and copolymers of the above.

In some instances, the shape memory foam may include a bioresorbable material such that resorption results in the formation of a biostable tissue matrix. Synthetic bioresorbable polymers may, for example, be selected from the following: (a) polyester homopolymers and copolymers such as polyglycolide (PGA; polyglycolic acid), polylactide (PLA; polylactic acid) including poly-L-lactide, poly-D-lactide and poly-D,L-lactide, poly(beta-hydroxybutyrate), polygluconate including poly-D-gluconate, poly-L-gluconate, poly-D,L-gluconate, poly(epsilon-caprolactone), poly(delta-valerolactone), poly(p-dioxanone), poly(lactide-co-glycolide) (PLGA), poly(lactide-codelta-valerolactone), poly(lactide-co-epsilon-caprolactone), poly(lactide-co-beta-malic acid), poly(beta-hydroxybutyrate-co-beta hydroxyvalerate), poly[1,3bis(p-carboxyphenoxy)propane-co-sebacic acid], and poly(sebacic acid-co-fumaric acid); (b) polycarbonate homopolymers and copolymers such as poly(trimethylene carbonate), poly(lactide-co-trimethylene carbonate) and poly(glycolide-co-trimethylene carbonate); (c) poly(ortho ester homopolymers and copolymers such as those synthesized by copolymerzation of various diketene acetals and diols; (d) polyanhydride homopolymers and copolymers such as poly(adipic anhydride), poly(suberic anhydride), poly(sebacic anhydride), poly(dodecanedioic anhydride), poly(maleic anhydride), poly[1,3-bis-(p-carboxyphenoxy)methane anhydride], and poly[alpha,omega-bis(p-carboxyphenoxy)alkane anhydride] such as poly[1,3-bis(p-carboxyphenoxy)propane anhydride] and poly[1,3-bis(p-carboxyphenoxy) hexane anhydride]; (e) polyphosphazenes such as aminated and alkoxy substituted polyphosphazenes; and (f) amino-acid-based polymers including tyrosine-based polymers such as tyrosine-based polyacrylates (e.g., copolymers of a diphenol and a diacid linked by ester bonds, with diphenols selected, for example, from ethyl, butyl, hexyl, octyl, and benzyl esters of desaminotyrosyl-tyrosine and diacids selected, for example, from succinic, glutaric, adipic, suberic, and sebacic acid), tyrosine-based polycarbonates (e.g., copolymers formed by the condensation polymerization of phosgene and a diphenol selected, for example, from ethyl, butyl, hexyl, octyl, and benzyl esters of desaminotyrosyl-tyrosine, tyrosine-based iminocarbonates, and tyrosine-, leucine- and lysine-based polyester-amides; specific examples of tyrosine-based polymers further include polymers that are comprised of a combination of desaminotyrosyl tyrosine hexyl ester, desaminotyrosyl tyrosine, and various di-acids, for example, succinic acid and adipic acid. Suitable materials include cross-linked polycarbonates and crosslinked polyethylene glycols.

In some instances, the shape memory foam may include thermoset polyurethanes that include oxidatively susceptible linkages in the soft segment, including but not limited to tertiary amines and polyethers. The shape memory foam may optionally include hydrolytically degradable soft segment components such as polycaprolactone, esters, and others.

A shape memory foam may have a thermal transition point (transition temperature) below which residual stress is maintained without a loading constraint. The thermal activation (which causes the shape memory) may be achieved with the desired material going through a semi-crystalline melt point or glass transition temperature between the first configuration and the expanded configuration. Several suitable thermal activation processes are known in the art and useful herein. In an example, the temperature activated memory shape foam may be formed for use as a medical device by first shaping a shape memory foam formed from a suitable material into its final expanded configuration; that is, the configuration that the shape memory shape foam will achieve once inserted into the left atrial appendage to provide the desired occlusive benefit. In this expanded configuration, the shape memory foam may generally have a diameter that will range from about 10 millimeters (about 0.39 inches) to about 50 millimeters (about 1.97 inches) and a length that will range from about 1 centimeter (about 0.39 inches) to about 5 centimeters (about 1.97 inches), although other diameters and lengths are within the scope of the present disclosure. Once this has been done, the foam may be heated above the transition temperature of the material; that is, the temperature at which a desired expansion will occur. Once the desired transition temperature has been achieved, the shape memory foam is held at a constant temperature and is re-shaped into an initial (unexpanded) configuration. This re-shaping is suitably done in a properly sized molding element and may be any suitable shape.

After insertion into the molding element, the temperature is reduced to a temperature below the transition temperature to set the new shape; for example, the temperature may be reduced to room temperature to set the new shape. Once this has been completed, the shape memory foam will remain in its first configuration until it is subjected to a temperature at or above the transition temperature, at which time it will expand into its expanded, or remembered, configuration. In some instances, exposure to water within the blood changes the glass transition temperature of the foam. As an example, the shape memory foam may have a dry T_{g} (glass transition temperature) that is above body temperature, and may have a wet T_{g}, after exposure to water, that is lower than body temperature.

In some cases, the shape memory polymers may include non-foamed versions of the polymers described herein with respect to making the expandable foams such as shape memory foams. Example of bio-compatible shape memory polymers include polymers made from poly(ε-caprolactone) (PCL), polyurethane (PU), poly (D, L-lactide) (PDLLA), PVA, ethylene vinyl acetate copolymer, (EVA) polymer blend, polymer composites, crosslinked polymers and supramolecular networks, among others. In some instances, shape memory polymers that may be used in creating the anchors described herein may include polyurethane, for example.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, the devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An implantable medical device, comprising:
a device body; and
an anchor including a shape memory polymeric component, the anchor secured relative to the device body and configured to secure the device body in a desired location;
wherein:
the shape memory polymeric component has a first configuration for initial implantation; and
the shape memory polymeric component has a second configuration subsequent to initial implantation.

2. The implantable medical device of claim 1, wherein the shape memory polymeric component comprises a shape memory foam.

3. The implantable medical device of claim 1, wherein the shape memory polymeric component comprises a shape memory polymer.

4. The implantable medical device of claim 1, wherein the shape memory polymeric component forms the anchor.

5. The implantable medical device of claim 1, wherein the shape memory polymeric component is disposed on the anchor.

6. The implantable medical device of claim 1, wherein the anchor further comprises a removable insertion aid.

7. The implantable medical device of claim 1, wherein the shape memory polymeric component changes from the first configuration to the second configuration as a result of a temperature increase of the shape memory polymeric component.

8. The implantable medical device of claim 1, wherein the shape memory polymeric component changes from the first configuration to the second configuration as a result of hydration.

9. The implantable medical device of claim 1, wherein the shape memory polymeric component changes from the first configuration to the second configuration as a result of active actuation.

10. An implantable medical device, comprising:
a device body; and
an anchor including a shape memory foam component, the anchor secured relative to the device body and configured to secure the device body in a desired location;
wherein:
the shape memory foam component has a compressed configuration for initial implantation; and
the shape memory foam component expands into an expanded configuration after implantation.

11. The implantable medical device of claim 10, wherein the shape memory foam component in its compressed configuration forms the anchor.

12. The implantable medical device of claim 11, wherein the shape memory foam component is compressed into a shape of a helical screw in its compressed configuration.

13. The implantable medical device of claim 11, wherein the shape memory foam component is adapted to accommodate an insertion aid therein, and the anchor is adapted to be inserted into tissue with the insertion aid positioned within the shape memory foam component.

14. The implantable medical device of claim 10, wherein the shape memory foam component is compressed onto the anchor.

15. An implantable medical device, comprising:
a device body; and
a shape memory anchor secured relative to the device body and configured to secure the device body in a desired location;
wherein:
the shape memory anchor has a first configuration for initial implantation; and
the shape memory anchor has a second configuration after implantation.

16. The implantable medical device of claim 15, wherein the first configuration comprises a linear configuration and the second configuration comprises a curved configuration.

17. The implantable medical device of claim 15, wherein the shape memory anchor includes one or more hooks in the second configuration.

18. The implantable medical device of claim 16, wherein the shape memory anchor has a T-shape or an L-shape in the second configuration.

19. The implantable medical device of claim 15, wherein the shape memory anchor comprises a coil.

20. The implantable medical device of claim 19, wherein the coil has a first pitch and/or diameter while in the first configuration and a second pitch and/or diameter while in the second configuration.
